(19) United States
(12) Patent Application Publication
(10) Pub. No.: US 2021/0322469 A1
(43) Pub. Date: Oct. 21, 2021

(54) ZINC AND COPPER FOR THE PROPHYLAXIS AND TREATMENT OF COVID-19

(71) Applicant: Regents of the University of Minnesota, Minneapolis, MN (US)

(72) Inventors: Fredericus J. van Kuijk, St. Paul, MN (US); Scott W. McPherson, Edina, MN (US)

(21) Appl. No.: 17/235,518

(22) Filed: Apr. 20, 2021

Related U.S. Application Data
(60) Provisional application No. 63/012,529, filed on Apr. 20, 2020.

(51) Int. Cl. A61K 33/34; A61K 31/375; A61K 31/355; A61K 31/047; A61K 9/00; A61K 33/30

(52) U.S. Cl. CPC A61K 33/34; A61K 31/375; A61K 33/30; A61K 31/047; A61K 9/0053; A61K 31/355

(57) ABSTRACT

This disclosure describes methods for prophylaxis and treatment of coronavirus in vulnerable populations and infected individuals. The methods include the administration of zinc and in some embodiments copper. In some embodiments, an amount of zinc is administered sufficient to raise serum zinc levels to 60 μg/dL. In some aspects, the coronavirus is the SARS-CoV-2 virus and/or the infection is COVID-19.

### RELATED APPLICATIONS

The present application claims the benefit of U.S. Provisional Patent Application No. 63/012,529, filed Apr. 20, 2020, which is incorporated herein by reference in its entirety.

### BACKGROUND

The novel coronavirus disease of 2019 (COVID-19), which can cause a severe respiratory syndrome in humans, results from infection by the SARS-CoV-2 virus. SARS-CoV-2 virus is an enveloped, positive-strand RNA virus that is about 80% identical to the SARS-CoV virus that was responsible for the Severe Acute Respiratory Syndrome (SARS) outbreak of 2002-2003. Safe and effective methods of preventing or treating COVID-19 are desperately needed.

### SUMMARY OF THE INVENTION

Disclosed herein is a method that includes the administration of zinc in combination with copper as an effective COVID-19 prophylaxis and treatment that may limit the severity of the disease in infected individuals (McPherson, S. W., J. E. Keunen, A. C. Bird, E. Y. Chew, and F. J. van Kuijk. 2020. Investigate Oral Zinc as a Prophylactic Treatment for Those at Risk for COVID-19. Am J Ophthalmol.).

The entry of SARS-CoV-2 into cells is dependent on the interaction between the spike glycoprotein of the virus and the peptidase domain of angiotensin converting enzyme 2 (ACE2) found on the surface of cells (Yan, R., Y. Zhang, Y. Li, L. Xia, Y. Guo, and Q. Zhou. 2020. Structural basis for the recognition of SARS-CoV-2 by full-length human ACE2. Science 367: 1444-1448). Zinc can inhibit the function of ACE2 (Speth R., C. E., Jean-Baptiste M., Joachim A., LInares A. 2014. Concentration dependent effects of zinc on angiotensin-converting enzyme-2. FASEB J 28: 1067). Latino, African American, and elderly populations have been shown to be disproportionally infected with SARS-CoV-2 and had more severe disease outcomes. These populations have been shown to have reduced levels of zinc intake and lower serum zinc levels (Briefel, R. R., K. Bialostosky, J. Kennedy-Stephenson, M. A. McDowell, R. B. Ervin, and J. D. Wright. 2000. Zinc intake of the U.S. population: findings from the third National Health and Nutrition Examination Survey, 1988-1994. J Nutr 130: 1367S-1373S; and Han, S., L. Wu, W. Wang, N. Li, and X. Wu. 2019. Trends in Dietary Nutrients by Demographic Characteristics and BMI among US Adults, 2003-2016. Nutrients 11), putting them at risk for worse outcomes following SARS-CoV2 infection. Recent data has borne this out showing that COVID-19 has reduced the life expectancy of Caucasian Americans by 0.68 years but Latino and African Americans by 2.10 and 3.05 years respectively (Andrasfay, T., and N. Goldman. 2021. Reductions in 2020 US life expectancy due to COVID-19 and the disproportionate impact on the Black and Latino populations. Proceedings of the National Academy of Sciences of the United States of America 118).

In comparing hospitalized intensive care (ICU) COVID-19 patients versus non-ICU patients we found that serum zinc levels were 17-20% lower at the time of admission in the ICU patients (63.5 mg/dL vs. 51.0 mg/dL). Additionally, we observed no COVID-19-related deaths among patients receiving zinc either prophylactically for age-related macular degeneration (AMD) or for treatment of AMD at the University of Minnesota, Adult Eye Clinic.

Disclosed herein therefore is zinc in combination with copper. In some embodiments, this combination of zinc and copper can be administered via a commercially available formulation referred to as the AREDS-2 formula (commercially available from Bausch & Lomb, PRESER VISION®). Administration of a combination of zinc and copper will act prophylactically to limit the severity of COVID-19 infection outcomes by raising serum zinc levels and will be effective in treating COVID-19 infected individuals by raising serum zinc levels.

This disclosure describes methods for prophylaxis for and treatment of coronavirus via the administration of zinc and copper. In some embodiments, the coronavirus is the SARS-CoV-2 virus and/or the infection is COVID-19. Zinc is administered in combination with copper. In some embodiments, this combination of zinc and copper can be provided by the AREDS-2 vitamin formulation (Bausch & Lomb, PRESER VISION®). Copper is included in order to prevent copper deficiency when higher doses of oral zinc are taken (Age-Related Eye Disease Study Research, G. 2002. The effect of five-year zinc supplementation on serum zinc, serum cholesterol and hematocrit in persons randomly assigned to treatment group in the age-related eye disease study: AREDS Report No. 7. J Nutr 132: 697-702). Although the AREDS-2 formulation contains the additional components vitamin C, vitamin E, Lutein, and Zeaxanthin, it is thought, but not relied upon that they aid in the efficacy against COVID-19. Additionally, the AREDS-2 formula has a well-known safety profile and contains the minimum amount of zinc necessary to improve outcomes in COVID-19.

In some embodiments zinc is administered to a patient to prevent or treat a coronavirus such as SARS-CoV-2. In some embodiments, zinc is administered to a patient to prevent, treat, or minimize the symptoms of a coronavirus such as SARS-CoV-2 or a COVID-19 infection. In some embodiments, zinc is administered to a patient to prevent, treat, or minimize the symptoms of a coronavirus such as SARS-CoV-2 or a COVID-19 infection by obtaining a particular serum level of zinc. In some embodiments zinc in combination with copper is administered to a patient to prevent or treat a coronavirus such as SARS-CoV-2. In some embodiments, zinc in combination with copper is administered to a patient to prevent, treat, or minimize the symptoms of a coronavirus such as SARS-CoV-2 or a COVID-19 infection. In some embodiments, zinc in combination with copper is administered to a patient to prevent, treat, or minimize the symptoms of a coronavirus such as SARS-CoV-2 or a COVID-19 infection by obtaining a particular serum level of zinc. In some embodiments, zinc is administered in the amount of at least 80 mg zinc oxide (or some other form of zinc, e.g., zinc gluconate, zinc sulfate, and zinc acetate) daily. In some embodiments, at least 80 mg zinc is administered in combination with at least 2 mg copper to achieve a sustained increase in serum zinc concentration.

The words “preferred” and preferably refer to embodiments of the invention that may afford certain benefits, under certain circumstances. However, other embodiments may also be preferred, under the same or other circumstances. Further, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to” a number (for example, up to 50) includes the number (for example, 50).

The term “in the range” or “within a range” (and similar statements) includes the endpoints of the stated range.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

This disclosure describes methods intended to treat and/or prevent infection with a coronavirus, the methods including the administration of at least 80 mg zinc (delivered from zinc oxide for example) and 2 mg copper (delivered from cupric oxide for example). In some embodiments, the coronavirus is the SARS-CoV-2 virus and/or the infection is COVID-19.

Treating or preventing coronavirus may include reducing or preventing coronavirus infection or the symptoms associated therewith or halting or slowing the progression of coronavirus infection or symptoms within a subject. Prophylactic treatment may be initiated before diagnosis or the development of symptoms of infection with coronavirus. Therapeutic treatment may be initiated immediately after diagnosis with coronavirus or after the development of symptoms of infection with coronavirus.

Treatment can be performed before, during, or after the diagnosis or development of symptoms of COVID-19 infection. Treatment initiated before a subject manifests symptoms of the infection with coronavirus may include, for example, treatment initiated after a subject has been exposed to coronavirus. In some embodiments, treatment may be initiated days, weeks, or even months and years before the subject is exposed to coronavirus. Treatment initiated days, weeks, or even months and years before the subject is exposed to coronavirus may be especially useful in vulnerable populations, such as those mentioned above.

Treatment initiated before or after the development of symptoms may result in decreasing the severity of one or more symptoms of coronavirus, or in completely removing one or more symptoms of coronavirus.

In one aspect, this disclosure describes a method of treating or preventing coronavirus in a subject, the method including administering zinc. The zinc may be administered via one of any number of forms, e.g., zinc gluconate, zinc sulfate, zinc acetate, zinc oxide, or any combination thereof. Zinc can be administered in combination with copper. The copper may be administered via one of any number of forms, e.g., cupric oxide, cupric sulfate, copper amino acid chelates, copper gluconate, or any combination thereof. Optionally, vitamin C, vitamin E, lutein, zeaxanthin, other vitamins, or combinations thereof may also be administered as well, but none of the optional components are required.

In some embodiments, at least 80 mg zinc, at least 90 mg zinc, or at least 100 mg zinc is administered daily. In some embodiments, up to 100 mg zinc, up to 150 mg zinc, up to 200 mg zinc, or up to 250 mg zinc is administered daily.

In some embodiments, zinc is administered in combination with copper. In some embodiments, the method includes administering at least 1.6 mg and up to 2.4 mg copper daily. In particular illustrative embodiments, the methods can include administering 2 mg copper daily.

In some embodiments, the methods can include administering a composition described in U.S. Pat. No. 8,603,522, which is incorporated by reference herein in its entirety. The composition may, in some embodiments, preferably be administered daily.

In illustrative embodiments, methods can include administering: 80 mg zinc, 500 mg vitamin C, 180 mg (400 IU) vitamin E, 10 mg lutein, 2 mg zeaxanthin, and 2 mg copper. In some embodiments, the method can be repeated daily or include daily administration of such compositions.

In another illustrative embodiment, the method includes administering PRESERVISION® AREDS-2 formula vitamins (Bausch+Lomb, Rochester, N.Y.). In some embodiments, the AREDS-2 formula vitamins can be administered daily.

As further described in Example 1, patients being treated for macular degeneration with the AREDS-2 formula vitamin composition at the time of the coronavirus outbreak in Minnesota unexpectedly showed reduced mortality compared to people in the same age group in April 2020, and this reduction in mortality (no reported deaths from SARS-CoV-2 to date) was maintained throughout the year until April 2021.

In another aspect, this disclosure describes administering zinc to a subject to treat or prevent infection with coronavirus or to halt or slow the progress of infection or symptoms within the subject. Serum zinc levels were reported to be lower by 17-20% in COVID-19 patients with more severe outcomes such as death or intubation in the ICU. This was more common in elderly, Latino, and African American patients.

In some embodiments, at least 80 mg zinc, at least 90 mg zinc, or at least 100 mg zinc is administered each day. In some embodiments, up to 100 mg zinc, up to 150 mg zinc, up to 200 mg zinc, or up to 250 mg zinc is administered daily.

In some embodiments, the dose of zinc is administered for at least 14 days, at least 21 days, at least 28 days, at least 35 days, at least 40 days, at least 45 days, at least 50 days, at least 60 days, at least 70 days, at least 80 days, at least 90 days, at least 100 days, at least 150 days, at least 200 days, or at least 5500 days prior to the subject being exposed to coronavirus.

In some embodiments, zinc is administered in amount sufficient to achieve a sustained serum value of zinc. In some embodiments, a minimum level of 60 mg/dL was found sufficient to prevent severe outcomes in COVID-19 patients.

Normal serum levels of zinc in humans are 60 μg/dL to 120 μg/dL. The AREDS-2 formula, which provides 80 mg of zinc daily, has been observed to increase serum zinc levels by 17% within one year. Such an increase may, however, be seen sooner. Thus, in some embodiments, zinc is administered to increase the serum zinc to at least a minimal normal level (approximately 60 μg/dL). In some embodiments, zinc is administered to increase the serum level of zinc in the patient to up to 17% above the maximum normal level (approximately 140 μg/dL). In some embodiments, the zinc is administered to increase serum zinc levels by up to 17% in the subject to whom the zinc is administered.

In some embodiments, the sustained serum value is maintained for at least 2 weeks, at least 4 weeks, at least 8 weeks, or at least 10 weeks. In some embodiments, the sustained serum value is maintained for up to 4 weeks, up to 8 weeks, up to 10 weeks, up to 20 weeks, up to 40 weeks, up to 80 weeks, up to 120 weeks, up to 140 weeks, up to 160 weeks, up to 180 weeks, or up to 785 weeks. In some embodiments, the sustained serum value may be maintained until all individuals are vaccinated for SARS-CoV-2, or until herd immunity against all SARS-CoV-2 variants is achieved.

Without wishing to be bound by theory, it is believed that the sustained increase in serum levels of zinc observed with patients being administered disclosed compositions, such as the AREDS-2 formula, as described in Example 2, may be protective against coronavirus and/or may halt or slow the progression of infection or symptoms within a subject. Disclosed compositions, such as the AREDS-2 formula does not prevent exposure to SARS-CoV-2, however it was unexpectedly found that elderly patients that were taking disclosed compositions, such as the AREDS-2 formulation and were exposed to SARS-CoV-2 remained asymptomatic and did not require hospitalization (it is noted that this is ongoing and as of April 2021, they are still asymptomatic and have not required hospitalization).

### Pharmaceutical Compositions

In one aspect, this disclosure describes a pharmaceutical composition that includes, as an active agent, zinc and a pharmaceutically acceptable carrier. The active agent may further include additional components in addition to zinc, as further described herein. In one aspect the pharmaceutical composition may also include copper.

In one aspect this disclosure describes a pharmaceutical composition that is similar to, includes, or is PRESERVISION® AREDS-2 formula vitamins (Bausch+Lomb, Rochester, N.Y.).

The pharmaceutical composition may be administered to a vertebrate, particularly a mammal, such as a human patient, companion animal, or domesticated animal, in a variety of forms adapted to the chosen route of administration. Exemplary formulations include those suitable for oral, rectal, vaginal, nasal, or parenteral (including subcutaneous, intramuscular, intraperitoneal, and intravenous) administration.

The pharmaceutically acceptable carrier can include, for example, an excipient, a diluent, a solvent, an accessory ingredient, a stabilizer, a protein carrier, or a biological compound. Non-limiting examples of a protein carrier includes keyhole limpet hemocyanin (KLH), bovine serum albumin (BSA), ovalbumin, or the like. Non-limiting examples of a biological compound which can serve as a carrier include a glycosaminoglycan, a proteoglycan, and albumin. The carrier can be a synthetic compound, such as dimethyl sulfoxide or a synthetic polymer, such as a polyalkyleneglycol. Ovalbumin, human serum albumin, other proteins, polyethylene glycol, or the like can be employed as the carrier. In a preferred embodiment, the pharmaceutically acceptable carrier includes at least one compound that is not naturally occurring or a product of nature.

The formulation may be conveniently presented in unit dosage form and can be prepared by any of the methods well-known in the art of pharmacy. In some embodiments, a method includes the step of bringing the active agent into association with a pharmaceutical carrier. In general, the formulations are prepared by uniformly and intimately bringing the active compound into association with a liquid carrier, a finely divided solid carrier, or both, and then, if necessary, shaping the product into the desired formulations.

A formulation of the present disclosure suitable for oral administration can be presented as discrete units such as tablets, troches, capsules, lozenges, wafers, or cachets, each containing a predetermined amount of the active agent as a powder or granules, as liposomes, or as a solution or suspension in an aqueous liquor or non-aqueous liquid such as a syrup, an elixir, an emulsion, or a draught. The tablets, troches, pills, capsules, and the like can also contain one or more of the following: a binder such as gum tragacanth, acacia, corn starch, or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it can further contain a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, sugar, and the like. A syrup or elixir can contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent can be incorporated into preparations and devices in formulations that may or may not be designed for sustained release.

A formulation suitable for parenteral administration may include a sterile aqueous preparation of the active agent, or dispersions of sterile powders of the active agent, which are preferably isotonic with the blood of the recipient. Parenteral administration (e. g., through an I. V. drip) is one form of administration. Isotonic agents that can be included in the liquid preparation include sugars, buffers, and sodium chloride. Solutions of the active agent can be prepared in water, optionally mixed with a nontoxic surfactant. Dispersions of the active agent can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the active agent, preferably by filter sterilization. Preferred methods for preparing powders include vacuum drying and freeze drying of the sterile injectable solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the active agents over a prolonged period can be achieved by including agents for delaying, for example, aluminum monostearate and gelatin.

A nasal spray formulation may include purified aqueous solutions of the active agent with preservative agents and isotonic agents. Such formulations are preferably adjusted to a pH and isotonic state compatible with the nasal mucous membranes. A formulation for rectal or vaginal administration can be presented as a suppository with a suitable carrier such as cocoa butter, or hydrogenated fats or hydrogenated fatty carboxylic acids.

### Administration

PRESERVISION® AREDS-2 formula vitamins (Bausch+Lomb, Rochester, N.Y.) are available over the counter (OTC) and are considered safe and effective for administration to mammals such as human beings. As such, the administration of PRESERVISION® AREDS-2 formula vitamins (Bausch+Lomb, Rochester, N.Y.) or similar compositions for the purpose of COVID-19 prophylaxis and treatment would also be considered safe. The formulations described herein may be administered as a single dose or in multiple doses.

Dosage levels of the active agent in the pharmaceutical compositions of this disclosure can be varied so as to obtain an amount of the active agent which is effective to achieve the desired therapeutic response for a particular subject, composition, and mode of administration, without being toxic to the subject. The selected dosage level may depend upon a variety of factors including the activity of the particular compound of the present disclosure employed, or the ester, salt or amide thereof, the route of administration, the time of administration, the rate of excretion of the particular compound being employed, the duration of the treatment, other drugs, compounds and/or materials used in combination with the aurone, the age, sex, weight, condition, general health and prior medical history of the subject being treated, and like factors known in the medical arts.

Dosages and dosing regimens that are known to be suitable for zinc and, if present, the other compounds, may serve as guideposts for developing suitable dosages and dosing regimens.

In some embodiments, the formulation may preferably be administered once daily, wherein the formulation includes all the components in a single daily dosage.

As an example, the formulation may be administered orally.

The present invention is illustrated by the following examples. It is to be understood that the particular examples, materials, amounts, and procedures are to be interpreted broadly in accordance with the scope and spirit of the invention as set forth herein.

### Illustrated Embodiments

Embodiment 1 is a method of treating or preventing coronavirus in a subject, the method comprising: administering a daily dosage of at least 80 mg zinc; and at least 2 mg copper to the subject.

Embodiment 2 is the method of embodiment 1, wherein the method further comprises administering an antioxidant.

Embodiment 3 is the method of any of embodiments 1 or 2, wherein the method comprises administering the zinc via zinc gluconate, zinc sulfate, zinc acetate, zinc oxide, or any combination thereof; and the copper via cupric oxide, cupric sulfate, copper amino acid chelates, copper gluconate, or any combination thereof.

Embodiment 4 is the method of any of embodiments 1 to 3, wherein the method comprises administering at least 100 mg zinc.

Embodiment 5 is the method of any of embodiments 1 to 4, wherein the method comprises administering not more than 250 mg zinc.

Embodiment 6 is the method of any of embodiments 1 to 5, wherein the method further comprises administering: at least 420 mg and up to 600 mg vitamin C, at least 400 IU and up to 540 IU vitamin E, and at least 0.04 mg and up to 40 mg of a lutein-zeaxanthin combination

Embodiment 7 is the method of any of embodiments 1 to 6, wherein the method further comprises administering 500 mg vitamin C, 180 mg (400 IU) vitamin E, 10 mg lutein, and 2 mg zeaxanthin.

Embodiment 8 is the method of any of embodiments 1 to 7, wherein the method comprises orally administering the daily dosage.

Embodiment 9 is a method of treating or preventing coronavirus in a subject, the method comprising: administering a daily dosage of at least 80 mg zinc, wherein the dose is administered for at least 14 days prior to the subject being exposed to coronavirus.

Embodiment 10 is the method of embodiment 9, wherein the method comprises administering up to 250 mg zinc.

Embodiment 11 is the method of any of embodiments 9 or 10, wherein the method comprises administering zinc in amount sufficient to achieve a sustained serum value of at least 60 μg/dL in the subject.

Embodiment 12 is the method of any of embodiments 9 to 11, wherein the sustained serum value is maintained for at least 2 weeks.

Embodiment 13 is the method of any of embodiments 9 to 12, wherein the method comprises administering at least 100 mg zinc.

Embodiment 14 is the method of any of embodiments 9 to 13, wherein the method further comprises administering: at least 420 mg and up to 600 mg vitamin C, at least 400 IU and up to 540 IU vitamin E, and at least 0.04 mg and up to 40 mg of a lutein-zeaxanthin combination

Embodiment 15 is the method of any of embodiments 9 to 14, wherein the method further comprises administering 500 mg vitamin C, 180 mg (400 IU) vitamin E, 10 mg lutein, and 2 mg zeaxanthin.

Embodiment 16 is the method of any of embodiments 9 to 15, wherein the method comprises orally administering the daily dosage.

Embodiment 17 is the method of any of embodiments 9 to 16, wherein the method does not comprise administering copper.

Embodiment 18 is the method of any of embodiments 9 to 17, wherein the method comprises administering at least 1.6 mg and up to 2.4 mg copper daily. Embodiment 19 is the method of any of embodiments 9 to 18, wherein the method comprises oral administration.

Embodiment 20 is a method of lessening symptoms of COVID-19 infection, the method comprising: administering a daily dosage of at least 80 mg zinc to achieve a sustained serum value of at least 60 μg/dL in the subject, wherein the sustained serum value is maintained for at least 2 weeks.

### EXAMPLES

### Example 1

The first coronavirus case in Minnesota was reported on Mar. 6, 2020. By Ap. 17, 2020, Minnesota reported more than 2,000 confirmed cases and 111 deaths; the actual infection rates were estimated to be 10-100 times higher than the confirmed infection rate. On that same date, 30% of Minnesota's cases were reported in persons aged 65 or greater; 25% of the cases were believed to be due to exposure in a congregate living setting; and the median age of the people who had died was 85 years old.

Wu, et al. (Wu, Z., and J. M. McGoogan. 2020. Characteristics of and Important Lessons From the Coronavirus Disease 2019 (COVID-19) Outbreak in China: Summary of a Report of 72314 Cases From the Chinese Center for Disease Control and Prevention. JAMA 323: 1239-1242) reported the case fatality rate in China (and, particularly, Hubei Province) for patients aged 70-79 years was 8%. A population of several hundred patients between the ages of 70 and 95 being treated for age-related macular degeneration were taking the PRESERVISION® AREDS-2 formula vitamins (Bausch+Lomb, Rochester, N.Y.) at the time of the coronavirus outbreak in Minnesota. The patients were being monitored closely by their care team (located at the University of Minnesota, Adult Eye Clinic). Surprisingly, as of Apr. 17, 2020, despite the high circulation of the virus in Minnesota and the high mortality rate for this age group, the care team reported that none of the patients seen regularly at the University of Minnesota, Adult Eye Clinic had died of coronavirus. As of Apr. 18, 2021 there continued to be no reported instances of COVID-19 fatalities in patients of the University of Minnesota, Adult Eye Clinic supplemented with the PRESERVISION® AREDS-2 formula vitamins (Bausch+Lomb, Rochester, N.Y.).

### Example 2

The interaction between the receptor binding domain of the spike glycoprotein of SARS-CoV-2 and the peptidase domain of angiotensin converting enzyme 2 (ACE2) has been noted as critical for viral entry into host cells (Yan, R., Y. Zhang, Y. Li, L. Xia, Y. Guo, and Q. Zhou. 2020. Structural basis for the recognition of SARS-CoV-2 by full-length human ACE2. Science 367: 1444-1448). Because of the strong link between ACE2 and SARS-CoV-2 infection, it was hypothesized by the instant inventors that inhibitors of ACE2 might act as therapeutic agents against COVID-19. The trace mineral zinc has already been demonstrated to be a safe inhibitor of ACE2 function that could potentially constrain the ability of SARS-CoV-2 to infect cells (Speth R., C. E., Jean-Baptiste M., Joachim A., LInares A. 2014. Concentration dependent effects of zinc on angiotensin-converting enzyme-2. FASEB J 28: 1067).

Based on the Age-Related Eye Disease Study (AREDS) and the AREDS 2 studies (Age-Related Eye Disease Study 2 Research, G. 2013. Lutein+zeaxanthin and omega-3 fatty acids for age-related macular degeneration: the Age-Related Eye Disease Study 2 (AREDS-2) randomized clinical trial. JAMA 309: 2005-2015) many, primarily elderly patients, are already taking zinc-containing supplements to limit the progression of their age-related macular degeneration. Normal serum levels of zinc are typically around 60 μg/dL but the AREDS-2 formula, which provides 80 mg of zinc daily, increases serum zinc by 17-20% (Age-Related Eye Disease Study Research, G. 2002. The effect of five-year zinc supplementation on serum zinc, serum cholesterol and hematocrit in persons randomly assigned to treatment group in the age-related eye disease study: AREDS Report No. 7. J Nutr 132: 697-702). Since this increase in serum zinc levels is the same as the difference in serum zinc levels that is associated with more severe COVID-19 outcomes the instant inventors believe that the increase in serum zinc levels may be viral-protective.

### Example 3

As of Apr. 18, 2021 556,381 cases of COVID-19 were reported in Minnesota with 7,020 deaths (6634 confirmed and 386 suspected COVID-19 cases). Fatalities in long-term care facilities were 61-66% (4332 fatalities) of the confirmed and total fatalities. As of Apr. 18, 2021 the instant inventors continue to see no fatalities in care-facility residents who are being treated for AMD at the University of Minnesota, Adult Eye Clinic. These patients are taking the PRESERVISION® AREDS-2 formula vitamins (Bausch+Lomb, Rochester, N.Y.) daily.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method of treating or preventing coronavirus in a subject, the method comprising:
administering a daily dosage of at least 80 mg zinc; and at least 2 mg copper to the subject.

2. The method of claim 1, wherein the method further comprises administering an antioxidant.

3. The method of claim 1, wherein the method comprises administering the zinc via zinc gluconate, zinc sulfate, zinc acetate, zinc oxide, or any combination thereof; and the copper via cupric oxide, cupric sulfate, copper amino acid chelates, copper gluconate, or any combination thereof.

4. The method of claim 1, wherein the method comprises administering at least 100 mg zinc.

5. The method of claim 1, wherein the method comprises administering not more than 250 mg zinc.

6. The method of claim 1, wherein the method further comprises administering:
at least 420 mg and up to 600 mg vitamin C,
at least 400 IU and up to 540 IU vitamin E,
at least 0.04 mg and up to 40 mg of a lutein-zeaxanthin combination

7. The method of claim 1, wherein the method further comprises administering 500 mg vitamin C, 180 mg (400 IU) vitamin E, 10 mg lutein, and 2 mg zeaxanthin.

8. The method of claim 1, wherein the method comprises orally administering the daily dosage.

9. A method of treating or preventing coronavirus in a subject, the method comprising:
administering a daily dosage of at least 80 mg zinc,
wherein the dose is administered for at least 14 days prior to the subject being exposed to coronavirus.

10. The method of claim 9, wherein the method comprises administering up to 250 mg zinc.

11. The method of claim 9, wherein the method comprises administering zinc in amount sufficient to achieve a sustained serum value of at least 60 μg/dL in the subject.

12. The method of claim 11, wherein the sustained serum value is maintained for at least 2 weeks

13. The method of claim 9, wherein the method comprises administering at least 100 mg zinc.

14. The method of claim 9, wherein the method further comprises administering:
at least 420 mg and up to 600 mg vitamin C,
at least 400 IU and up to 540 IU vitamin E,
at least 0.04 mg and up to 40 mg of a lutein-zeaxanthin combination

15. The method of claim 9, wherein the method further comprises administering 500 mg vitamin C, 180 mg (400 IU) vitamin E, 10 mg lutein, and 2 mg zeaxanthin.

16. The method of claim 9, wherein the method comprises orally administering the daily dosage.

17. The method of claim 9, wherein the method does not comprise administering copper.

18. The method of claim 9, wherein the method comprises administering at least 1.6 mg and up to 2.4 mg copper daily.

19. The method of claim 9, wherein the method comprises oral administration.

20. A method of lessening symptoms of COVID-19 infection, the method comprising:
administering a daily dosage of at least 80 mg zinc to achieve a sustained serum value of at least 60 μg/dL in the subject,
wherein the sustained serum value is maintained for at least 2 weeks.
